(19) United States
(12) Patent Application Publication
(10) Pub. No.: US 2021/0077002 A1
(43) Pub. Date: Mar. 18, 2021

(54) Blood Collection And Processing Device

(71) Applicant: National University of Singapore, Singapore (SG)

(72) Inventor: Luke P. LEE, Singapore (SG)

(21) Appl. No.: 16/620,439

(22) PCT Filed: Jun. 7, 2018
(86) PCT No.: PCT/SG2018/050285

§ 371 Date: Dec. 6, 2019

Related U.S. Application Data
(60) Provisional application No. 62/516,731, filed on Jun. 8, 2017.

(51) Int. Cl. A61B 5/15; B01L 3/00; B01L 7/00

(52) U.S. Cl. CPC A61B 5/150755; B01L 2400/049; B01L 3/502753; B01L 7/52; A61B 5/150022; A61B 5/150068; A61B 5/150114; A61B 5/150412; A61B 5/150984; B01L 2300/0681; B01L 2300/0819; B01L 2300/0832; B01L 2300/0864; B01L 2300/087; B01L 2400/0406; B01L 3/50255

(57) ABSTRACT

A blood sample collection device can provide for collection, storage, amplification, and analysis of a blood sample in a simple, disposable device. The device can include a collection zone for drawing (e.g., via a needle array) or receiving a blood sample, a processing zone for storing and processing blood cells of the blood sample, and a filtration zone for selectively directing the blood cells of the blood sample to an array of wells within the processing zone while leading blood plasma away from the array of wells. Within the wells, which may be prepared to include materials for amplification and various assays, the blood cells can undergo amplification. Externally applied light and/or heat can facilitate amplification. A transparent or translucent window or portion of the collection device can allow an analysis device to take measurements of the array of wells without removing the sample.

### TECHNICAL FIELD

The present disclosure relates to the sampling and analysis of biological materials generally and more specifically to collecting, amplifying, and analyzing blood samples.

### BRIEF DESCRIPTION OF THE DRAWINGS

The specification makes reference to the following appended figures, in which use of like reference numerals in different figures is intended to illustrate like or analogous components.

FIG. 1 is schematic diagram depicting a blood collection, amplification, and analysis system according to certain aspects of the present disclosure.

FIG. 2 is a cutaway elevation view depicting a collection device with a multi-layered filtration zone according to certain aspects of the present disclosure.

FIG. 3 is a cutaway elevation view depicting a collection device with a branched filtration zone according to certain aspects of the present disclosure.

FIG. 4 is a partially see-through axonometric view depicting a round collection device according to certain aspects of the present disclosure.

FIG. 5 is a partially see-through top view of the round collection device of FIG. 4 according to certain aspects of the disclosure.

FIG. 6 is a side, elevation view of the round collection device of FIG. 4 according to certain aspects of the present disclosure.

FIG. 7 is a top cutaway view of the round collection device of FIG. 4 taken along line A:A of FIG. 6 according to certain aspects of the present disclosure.

FIG. 8 is a top view of an array of wells of a processing zone of a collection device according to certain aspects of the present disclosure.

FIG. 9 is a partial cut-away axonometric view depicting the round collection device of FIG. 4 according to certain aspects of the present disclosure.

FIG. 10 is a partially see-through axonometric view depicting a square collection device according to certain aspects of the present disclosure.

FIG. 11 is a partially see-through top view of the square collection device of FIG. 10 according to certain aspects of the disclosure.

FIG. 12 is a side, elevation view of the square collection device of FIG. 10 according to certain aspects of the present disclosure.

FIG. 13 is a top cutaway view of the square collection device of FIG. 10 taken along line B:B of FIG. 12 according to certain aspects of the present disclosure.

FIG. 14 is a schematic side view depicting an analysis system for detecting radiation emitted from a blood sample collection device prepared with multiple primers according to certain aspects of the present disclosure.

FIG. 15 is a flowchart depicting a process for collecting, amplifying, and analyzing a sample using a blood sample collection device according to certain aspects of the present disclosure.

FIG. 16 is a combination image depicting a bottom view of a collection device and a top view of an analysis device according to certain aspects of the present disclosure.

FIG. 17 is a bottom view of a collection device featuring a recognizable pattern according to certain aspects of the present disclosure.

### DETAILED DESCRIPTION

Certain aspects and features of the present disclosure relate to a device for collecting and processing blood samples using a quick, efficient, disposable capture and processing device. The capture and processing device can collect, amplify, and facilitate analysis of biological materials, such as genetic materials, containing in blood. The device can include a needle array containing one or more needles suitable for puncturing a user's epidermis and facilitating blood flow into the capture and processing device. Blood can flow through progressively smaller apertures to filter out plasma and allow blood cells to be captured within wells of the device. Any combination of hydrophilic surfaces, gravity, and capillary action can facilitate capturing the blood in the wells. The wells can be prepared to include probes, primers, or other materials for facilitating amplification and/or analysis of the collected blood, or such materials can optionally be added after blood collection. The capture and processing device can be small and disposable, allowing quick collection of blood samples and rapid amplification and/or analysis of the samples. In some cases, once a sample has been collected, the entire device can be placed in a thermal cycler to perform polymerase chain reaction (PCR) amplification without the need to transfer the sample to another receptacle. After undergoing amplification, the entire device can be placed in an instrument for analysis, such as through measurement of fluorescence. In some cases, the wells can include a plasmonic layer protected by a coating (e.g., SiO₂ or Al₂O₃), which can help avoid fluorescent quenching during analysis. The entire device can provide a quick, accurate, easy, efficient, and disposable solution for analyzing genetic material in a blood sample. The device enables new, minimally-invasive methods for collecting and processing blood samples for various diagnostic or research uses.

In use, the device is able to capture blood samples (e.g., collected from a patient's digit, such as a thumb), amplify any nucleic acids (NA)s present in the sample using polymerase chain reaction (PCR) amplification, and optionally detect one or more deoxyribonucleic acid (DNA) analyte(s) of interest, such as using fluorescent probes. The device can be hand-held in size, such as approximately one to four centimeters in diameter, approximately 1.5-2 cm in diameter, or any other suitable size. The device can be any suitable shape, such as round or square. The device can be made from materials suitable for exposure to the thermal cycling and temperatures used for PCR amplification. For example, the device can be made using materials suitable for withstanding temperatures between at or below approximately 50° C. or potentially at or below approximately 4° C., as well as at or above approximately 96° C. or potentially as at or above approximately 98° C.

The sample collection device disclosed herein can be used to collect genetic material from a blood sample. The sample collection device can include a collection zone, a filtration zone, and a processing zone. The collection zone can include a needle array for initiating blood collection. The needle array can include one or more needles designed to facilitate collecting blood from a user. The needle or needles can have a length designed to inhibit pain while still allowing blood to be drawn. The needle or needles can be hypodermic or solid, such as with grooves to facilitate blood flow into the sample collection device. The collection zone can include a set of sloped surfaces, grooves, and/or apertures for directing collected blood into the filtration zone. The filtration zone can include a number of passageways of sequentially decreasing diameter for directing blood cells towards the individual wells of the processing zone. In some cases, features of the collection zone and/or filtration zone can separate blood plasma from blood cells, directing the blood cells towards the wells of the processing zone while directing blood plasma towards collection areas. For example, the passageways of the filtration zone can allow blood cells to become trapped within the passageways and be directed towards the wells of the processing zone while the blood plasma is allowed to flow away into other areas of the sample collection device.

The processing zone can include an array of individual wells. Blood cells exiting the filtration zone of the sample processing device can be directed into the wells of the processing zone and become entrapped within. When entrapped within the individual wells, the blood cells can undergo any suitable preparation (e.g., lysing and DNA amplification), prior to analysis (e.g., visual detection of fluorescent probes). The individual wells can be cylindrical in shape, although they may alternatively be of any other suitable shape, such as rectangular, square, spherical, cubical, pyramidal, or conical. In some cases, the individual wells can be generally cylindrical in shape with a hexagonal cross section. The individual wells can be prepared to include any number of materials necessary for amplification and/or analysis of the entrapped samples, such as PCR reagents and DNA probes. The array of wells can provide an array for digital PCR analysis. In some cases, each well in the array of wells can include the same pre-applied materials for amplification and/or analysis. In some cases, the array of wells can include multiple subsets of wells (e.g., one or more wells), with each subset prepared with different materials, allowing different amplification and/or analysis to be run from a single collection event. In some cases, a subset of wells can include more than one well, allowing simultaneous, parallel analysis of entrapped samples. Subsets of wells can be arranged randomly throughout the array of wells or can be divided according to any suitable dimension. For example, subsets of wells can be divided based on angular position around the array of wells, or subsets of wells can be divided based on radial distance from the center of the array of wells, although other dimensional divisions can be used.

When subsets of wells are used for performing different analysis, an alignment/orientation system can be used to ensure the location of the subset of wells is known to the researcher or the system performing the analysis. The alignment/orientation system can be mechanical, visual, or automatic. For example, a mechanical alignment/orientation system can include the use of keyed features that fit within grooves of the analysis machine to ensure the alignment/orientation of the sample collection device is known when the analysis machine performs its readings. Mechanical alignment/orientation can include the use of other features, such as magnets designed to facilitate placing the sample collection device pm the analysis machine in a known alignment/orientation. As another example, visual alignment/orientation can be achieved by having a user line up indicators on the sample collection device with corresponding indicators on the analysis machine. In some cases, the alignment/orientation system can be automatic. An automatic system can include using sensors of the analysis machine to determine the alignment/orientation of the sample collection device. For example, the analysis machine can include cameras or magnetic sensors to detect mechanical or visual alignment/orientation features of the sample collection device. In some cases, the sample collection device can include alignment/orientation features in the individual wells designed to produce a recognizable pattern when the analysis machine reads the sample collection device. In such cases, the recognizable pattern can be used by a software module to determine the alignment/orientation of the sample collection device.

For example, in some cases, different primers specific for different DNA sequences can be immobilized within separate regions of (e.g., subsets of) the array of wells. Such a multiplexed collection device can undergo normal collection and amplification procedures and then undergo a multiplex analysis procedure, wherein the analysis machine is capable of detecting and/or measuring the presence of different DNA sequences based on measurements taken of the separate regions of the sample chamber. The separate regions can be overlapping with one another or can be distinct from one another. Primers can be immobilized in the separate regions during manufacturing, immediately prior to sample collection, or prior to amplification.

In some cases, the sample collection device can include an array of wells having approximately 100,000 wells, although any suitable number of wells can be used, including at least 1 or more wells. The apertures of the collection zone, the passageways of the filtration zone, and the wells of the processing zone can have any suitable dimensions. In some cases, the wells of the processing zone can be on the order of a few to tens of microns in diameter. In some cases, the wells of the processing zone can have diameters of approximately 1-100 μm, 2-100 μm, 10-100 μm, 2-50 μm, 2-10 μm, 10-50 μm, 10-20 μm, 5-10 μm, or approximately 5 or 10 μm, although other sizes can be used. In some cases, all wells of an array of wells can have the same or approximately same diameters, although that need not be the case.

The diameters of the passageways of the filtration zone can be any suitable size for filtering the blood and directing the blood cells towards the wells of the processing zone. The diameters of the passageways of the filtration zone can vary from larger to smaller across the height of the filtration zone, from furthest to the processing zone to nearest to the processing zone. In some cases, the smallest passageways of the filtration zone can be at or approximately 1, 2, 3, 1-3, or 1-5 times the diameter of the individual wells thereunder. In some cases, the passageways can increase in diameter up to approximately 500-500,000 μm, 500-100,000 μm, 500-10,000 μm, 500-2,000 μm, or 1,000 μm. The apertures of the collection zone can be of any suitable size for passing blood therethrough, such as on the order of a few millimeters or tens of millimeters, although other sizes can be used.

In some cases, the various blood-contacting surfaces of the sample collection device can be coated in, treated with, or made of hydrophilic materials. Gravity and/or capillary action can facilitate movement of blood cells into the wells of the processing zone. In some cases, a vacuum can be applied to facilitate drawing the blood cells into the wells. A vacuum can be externally applied, such as when the sample collection device is inserted into a processing device (e.g., PCR machine or thermal cycler). In some cases, a vacuum can be self-applied when a user depresses the sample collection device to give a sample. In such cases, applying pressure through the sample collection device, such as through a digit (e.g., thumb) can induce deformation of deformable features within the sample collection device which, when pressure is released, can revert to their original shape to induce a vacuum that facilitates drawing the blood sample through the filtration zone and the blood cells into the processing zone

The surfaces of the wells can be made of or can include a layer of a material suitable for reflecting, amplifying, resonating, or otherwise improving the radiation emitted within the sample chamber (e.g., by DNA probes) that is desired to be measured in an analysis step. The plasmonic layer is integrated for rapid and accurate photonic PCR, which allows LED light pulses to control for effective heat-transfer and PCR thermal cycle. For example, it is often desirable for light to be measured during an analysis step (e.g., fluorescent analysis), and thus the surfaces of the wells can be made of materials designed to inhibit fluorescent quenching. The surfaces of the wells can be made of or can include a layer of plasmonic material. The plasmonic material can be selected from any suitable plasmonic material, including gold, aluminum, or other such plasmonic materials. The interior surface of each well can include a layer or thin film of metallic nanoparticles (e.g., gold or aluminum). In some cases, the interior surface of a well can be prepared by depositing a thin film of metal on the surface and annealing the metal sufficiently to cause the metal to form beads on the surface. In some cases, the bottom surface of the well can be made of a transparent or sufficiently translucent material to facilitate transmission of light into and out of the well.

In some cases, the surfaces of the wells can be further covered in a protective layer. The protective layer can be located over the plasmonic material. The protective layer can be made of any suitable material, such as Silicon Dioxide (SiO₂) or Aluminum Oxide (Al₂O₃). The protective layer can help inhibit fluorescence quenching. The protective layer can be directly exposed to the blood cells when the blood cells are entrapped within the wells.

After a sample has been collected, the samples can undergo PCR amplification. PCT amplification can include thermal cycling, which can be accomplished through the use of light emitting diodes (LEDs) directed towards the array of wells. LEDs can accomplish the desired thermal cycling to amplify the samples. After amplification, the samples can be analyzed using any suitable analysis device, such as through an automated digital PCR reader. The analysis device can detect markers (e.g., fluorescent markers) in individual wells of the array of wells.

The collection device can include a window suitable for allowing radiation to pass from the individual wells to an analysis machine. In cases where light is being measured, the window can be an optically transparent window. In cases where other forms of radiation are being measured, the window can be transparent to the specific form of radiation being measured without necessarily being optically transparent. The window can be placed in any suitable location, such as the bottom of the device (e.g., closest the individual wells). The window can occupy up to 100% of the bottom of the device (e.g., the entire bottom of the device can be made from an optically transparent material), or can occupy less than 100% of the bottom of the device (e.g., the window is surrounded by a bezel of the material of the bottom of the device). As used herein, the term “transparent” can include 100% transparent as well as sufficiently translucent to carry out its intended purpose (e.g., to facilitate reading digital PCR from the individual wells).

The sample collection device can be formed as a single disposable unit. The entire sample collection device can be processed by a PCR machine (e.g., thermal cycler) and analyzed by an appropriate analysis device, without needing to separate any sample chambers (e.g., wells) from the remainder of the sample collection device. However, in other cases, one or more of the collection zone, filtration zone, and processing zone can be separable. For example, the processing zone can be separable from the collection zone and filtration zone, such as to allow the processing zone to be processed in a PCR machine without the collection zone and filtration zone.

A sample collection device can be individually provided in a sterile container for individual use. In some cases, multiple sample collection devices can be deployed simultaneously and can be individually uncovered for sample collection and recovered for temporary storage while other samples are being collected. For example, a large population can be sampled rapidly by carrying a clipboard of sample collection devices through the population and uncovering a new sample collection device at each individual of the population for sample collection prior to re-covering the sample collection device, notating any necessary identification markings, and moving on to the next individual. After sample collection has completed, the entire array of sample collection devices can be processed collectively or individually.

In some cases, the sample collection device can be small enough to be processed (e.g., amplified and analyzed) using a mobile device, such as a suitable mobile phone with any necessary attachments or adaptors. In an example, a sample collection device can be placed in or on a processing device capable of thermal cycling and digital PCR reading, and the processing device can be couplable (e.g., wired or wirelessly) to a mobile device (e.g., a computer, a tablet, or a smartphone) for rapid analysis. In some cases, the processing device can be provided power from the mobile device.

In an example use case, a sample can be collected by having a patient depress a digit (e.g., thumb) into the collection zone. The digit can be punctured by one or more needles of the needle array and blood can flow into the collection zone. In some cases, blood can be directly added to the collection zone, such as from a pipette or other source. Blood can flow into the filtration zone of the collection device, such as through apertures in the collection zone. Blood cells can pass through the passageways in the filtration zone, while excess plasma can flow away into excess regions of the sample collection device. Blood cells can pass from the filtration zone into individual wells of the processing zone of the collection device.

The sample collection process can be performed once or repeated multiple times. In some cases, the collection zone can be sealed or otherwise covered after sample collection. In some cases, the collection zone can be sealed prior to sample collection, such as to maintain sterility. PCR reagents can be added to the array of wells of the device (e.g., prior to sealing the collection zone, prior to assembly of the device, or through another opening). In some cases, PCR reagents can be added to the wells prior to sample collection, such as during manufacture or immediately prior to sample collection. The entire collection device can be treated according to standard PCR protocols to amplify the DNA collected in the wells. The collection device can be manually processed or can be placed in an automated PCR machine (e.g., a thermal cycler). In some cases, the collection device can be placed into a custom-fit PCR machine or a custom-fit adaptor for a standard PCR machine.

After PCR amplification is complete, the entire device can be analyzed, such as through measuring fluorescence of a fluorescent probe that is specific to the nucleotide sequence of interest. In some cases, analysis can be performed manually or with an automated analysis machine. An automated analysis machine can include a sensor suitable for detecting radiation emitted from the probe in question, such as electromagnetic radiation in the form of fluorescent light. In some cases, other forms of radiation can be detected, such as nuclear radiation (e.g., from a radioactive probe), heat, visible or non-visible light, or other radiation. If desired, other measurements may be taken.

In some cases, the collection device can be also used to amplify and detect genetic material found in a blood sample with the use of a lysing agent to lyse the cellular membranes of those organisms. In some cases, the lysing agent can be added to the blood sample near or at the end of sample collection, or can be introduced directly to the wells, such as during manufacture, immediately prior to sample collection, during sample collection (e.g., via the filtration zone of an alternate inlet), or after sample collection.

These illustrative examples are given to introduce the reader to the general subject matter discussed here and are not intended to limit the scope of the disclosed concepts. The following sections describe various additional features and examples with reference to the drawings in which like numerals indicate like elements, and directional descriptions are used to describe the illustrative embodiments but, like the illustrative embodiments, should not be used to limit the present disclosure. The elements included in the illustrations herein may not be drawn to scale.

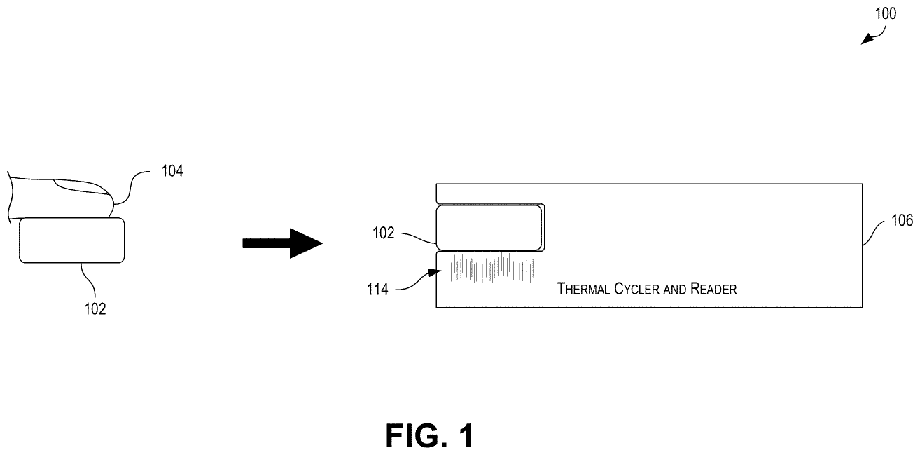

FIG. 1 is schematic diagram depicting a blood collection, amplification, and analysis system 100 according to certain aspects of the present disclosure. The analysis system 100 includes a collection device 102. A user can depress a digit 104 (e.g., finger or thumb) onto the collection device 102 to extract a blood sample. A portion of the blood sample can pass into the collection device 102, through a filtration zone, and into a processing zone. Blood cells from the blood sample can be directed into individual wells within the processing zone of the collection device 102. In some cases, a blood sample can be provided directly to the collection device 102, such as through a pipette or other fluid transfer device.

After the blood sample has been collected in the collection device 102, the collection device 102 can be thermally cycled to perform PCR amplification and read using optical sensors (e.g., fluorescent readers). In some cases, the collection device 102 can be placed within a single processing device 106 capable of performing both thermal cycling and optical reading.

Optionally, PCR reagents can be added to the wells of the collection device 102 if PCR reagents were not previously placed therein. Within the processing device 106, the entire collection device 102 or at least the processing zone of the collection device 102 can be heated and cooled as necessary to perform standard PCR amplification of the genetic material collected within, without needing to remove the sample from the collection device 102. Thermal cycling can be controlled through the application of light, such as from light emitting diodes (LEDs), such as photothermal LEDs.

After PCR amplification is complete, the collection device 102 can be optically read. The processing device 106 can detect radiation 114, such as visible light. Radiation 114 (e.g., visible or non-visible light), can be emitted from DNA probes and collected by sensors of the processing device 106.

In some cases, PCR amplification and optical reading can occur in a single processing device 106, as depicted in FIG. 1. In other cases, the collection device 102 can be placed within a PCR machine for PCR amplification and thereafter transferred to an analysis machine for optical reading.

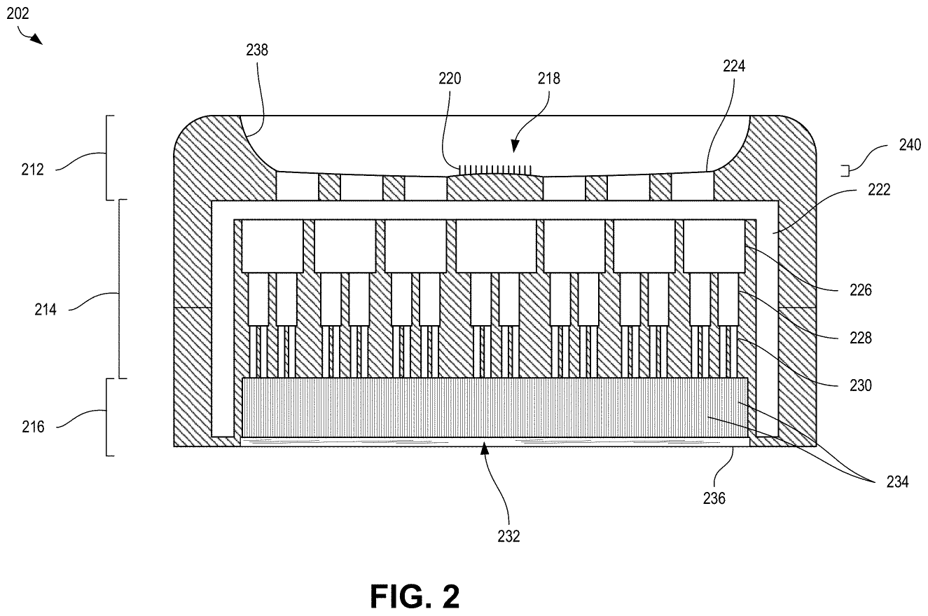

FIG. 2 is a cutaway elevation view depicting a collection device 202 with a multi-layered filtration zone 208 according to certain aspects of the present disclosure. The collection device 220 is not necessarily drawn to scale and features may be exaggerated for explanation purposes. The collection device 202 can include a collection zone 212, a filtration zone 214, and a processing zone 216. A blood sample can be initially provided at the collection zone 212. Gravity, vacuum force, and/or capillary action can facilitate movement of blood cells from the collection zone 212 towards the processing zone 216. The filtration zone 214 can facilitate separating blood cells from the blood sample, allowing blood plasma to flow into an excess reservoir 222.

The collection zone 212 can include a recess 238 into which a digit can be easily placed. The recess 238 can have walls that help retain the blood sample within the collection device 202. The collection zone 212 can include an array of needles 218 placed to contact and facilitate drawing of blood from a digit placed in the recess 238. The array of needles 218 can include a single needle 220 or multiple needles 220. The needle(s) 220 of the array of needles 218 can extend away from the bottom wall of the recess 238 a needle length 240. The needle length 240 can be selected to be sufficiently long so as to facilitate drawing of blood, however sufficiently short so as to minimize or eliminate pain associated with drawing of blood. The collection zone 212 can include apertures 224 that allow the blood sample to drain from the collection zone 212 into the filtration zone 214.

Blood sample entering the filtration zone 214 can enter an array of passageways 226, 228, 230 extending from adjacent the collection zone 212 to adjacent the processing zone 216. The passageways 226, 228, 230 can include openings having relatively wide diameters adjacent the collection zone 212 and openings having relatively small diameters adjacent the processing zone 216. In some cases, passageways 226, 228, 230 can be include of multiple layers of passageways. Any suitable number of layers can be used.

As depicted in FIG. 2, a three-layer filtration zone 214 is used, although a filtration zone 214 having one, two, or more than three layers can be used. A three-layer filtration zone 214 can include a set of proximal passageways 226, a set of middle passageways 228, and a set of distal passageways 230. The proximal passageways 226 can have a relatively large diameter, the distal passageways 230 can have a relatively small diameter, and the middle passageways 228 can have a diameter between that of the proximal passageways 226 and distal passageways 230. As the passageways 226, 228, 230 begin to fill with the blood sample, the blood cells of the blood sample can continue to pass through the passageways 226, 228, 230 until they reach the end of the distal passageway 230 (e.g., the bottom of the filtration zone 208). At least some blood plasma, however, can wash over the passageways 226, 228, 230 and into the excess reservoir 222. The excess reservoir 222 can be positioned anywhere within the collection device 202 where it will not interfere with sample analysis. For example, the excess reservoir 222 can be located radially further away from the center of the collection device 202 than the array of wells 232.

In some cases, each proximal passageway 226 can fluidly couple to multiple distal passageways 230. For example, a single proximal passageway 226 may fluidly couple to at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 128, 256, 512, or 1024 distal passageways 230.

Blood cells that have passed through the passageways 226, 228, 230 can collect in individual wells 234 of the array of wells 232. In some cases, an array of wells 232 can include a single well. However, the array of wells 232 can include any number of wells 234, including two or more wells 234. In some cases, the number of wells 234 can be in excess of 10,000 or 100,000. Each well 234 can have an interior surface (e.g., wall) and a bottom. The bottom of the well 234 can be transparent or sufficiently translucent. The bottom of the well 234 can be or can abut a window 236. The window 236 can be a transparent or sufficiently translucent material suitable for facilitating analysis of radiation passing from the wells 234 towards an analysis device (e.g., positioned below the collection device 202). In some cases, the window 236 can be a separate material from the main body of the collection device 202, or in some cases the main body of the collection device 202, or at least a portion thereof, can be made of a suitable transparent or sufficiently translucent material.

The collection device 202 can be made of any suitable number of parts. In some cases, the collection zone 212 can be separable from the filtration zone 214 and/or the processing zone 216. In some cases the filtration zone 214 can be separable from the collection zone 212 and/or the processing zone 216. In some cases, the processing zone 216 can be separable from the collection zone 212 and/or the filtration zone 214. Any combination of the zones 212, 214, 216 can be formed monolithically (e.g., of a single continuous body).

Before use, materials can be deposited within the wells 234 of the array of wells 232, such as PCR reagents, DNA probes, or other suitable materials. In some cases, a vacuum pump can be used to pre-apply a vacuum and/or to degas any materials that are included in the array of wells 232 to facilitate drawing the blood cells into the wells 234 when a sample is taken.

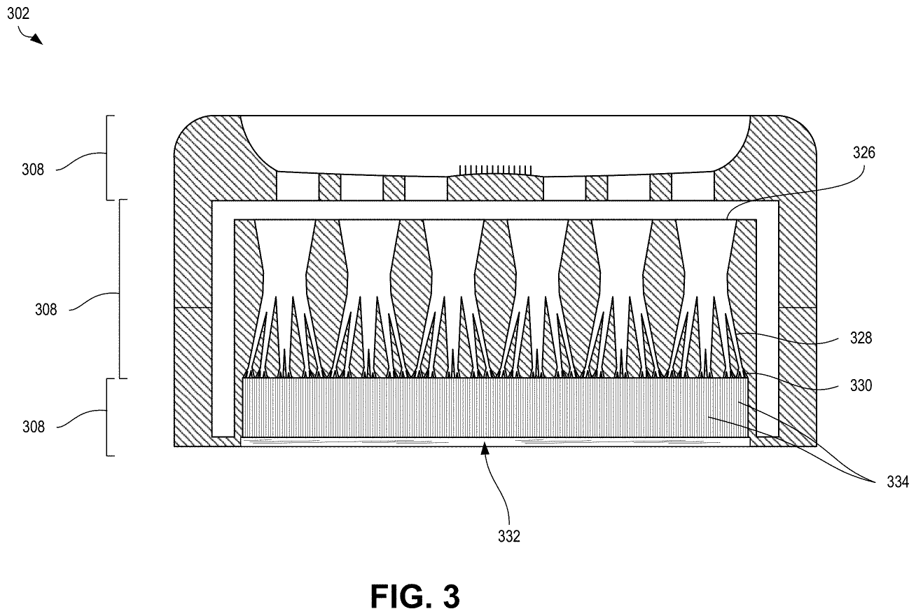

FIG. 3 is a cutaway elevation view depicting a collection device 302 with a branched filtration zone 308 according to certain aspects of the present disclosure. The collection device 320 is not necessarily drawn to scale and features may be exaggerated for explanation purposes. The collection device 302 can include a collection zone 312, a filtration zone 314, and a processing zone 316. The collection zone 312 and processing zone 316 can be similar to collection zone 212 and processing zone 216 of the collection device 202 of FIG. 2.

Blood sample entering the filtration zone 314 can enter an array of branching passageways extending from adjacent the collection zone 312 to adjacent the processing zone 316. The passageways can have proximal ends 326 with relatively wide diameters adjacent the collection zone 312 and distal ends 330 with relatively small diameters adjacent the processing zone 316.

As depicted in FIG. 3, the passageways begin at their proximal ends 326 and proceed to decrease in diameter as they extend towards their distal ends 330. Any number of intermediate passageways 328 can branch off the initial passageway or off subsequent intermediate passageways 328. Any number of distal ends 330 can branch off from the initial passageway or any of the intermediate passageways 328. In some cases, each proximal end 326 can fluidly couple to multiple distal ends 330. For example, a single proximal end 326 may fluidly couple to at least 2, 4, 6, 8, 10, 12, 14, 16, 18, 20, 22, 24, 26, 28, 30, 32, 34, 36, 38, 40, 42, 44, 46, 48, 50, 52, 54, 56, 58, 60, 62, 64, 128, 256, 512, or 1024 distal ends 330. Each distal end 330 can deliver blood cells to one or more wells of the array of wells.

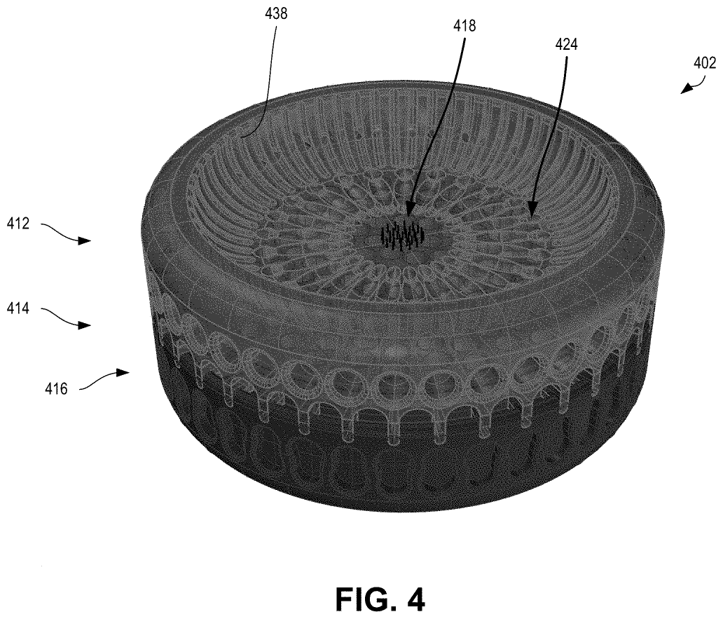

FIG. 4 is a partially see-through axonometric view depicting a round collection device 402 according to certain aspects of the present disclosure. The collection device 402 can include a collection zone 412, a filtration zone 414, and a processing zone 416. The collection zone 412 can include a recess 438 suitable for placement of a digit, such as a finger or thumb. A needle array 418 can be seen in the center of the collection zone 412, although it may be placed off-center in some cases. Various apertures 424 of different shapes and dimensions are seen in the collection zone 412. The collection device 402 further includes optional, external ornamental apertures and shapes.

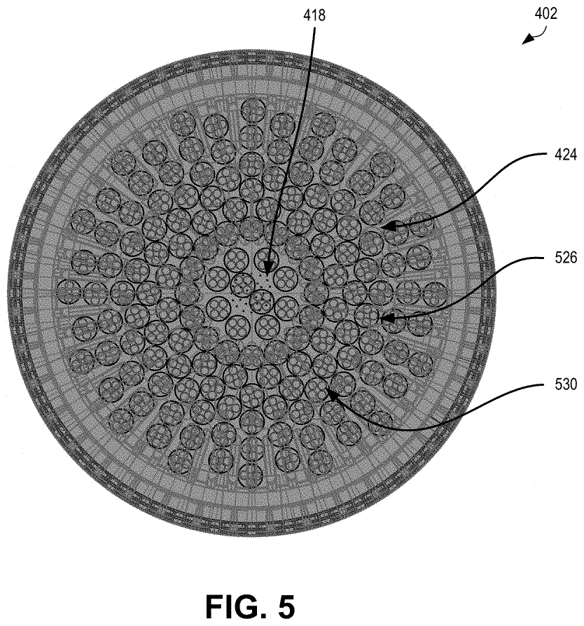

FIG. 5 is a partially see-through top view of the round collection device 402 of FIG. 4 according to certain aspects of the disclosure. The needle array 418 is seen centrally located. Numerous apertures 424 in the collection zone are depicted. Also visible in see-through are numerous proximal passageways 526 and distal passageways 530 of the filtration zone. The apertures 424, and passageways 526, 530 can be arranged radially about the center of the collection device 402, or can be otherwise arranged.

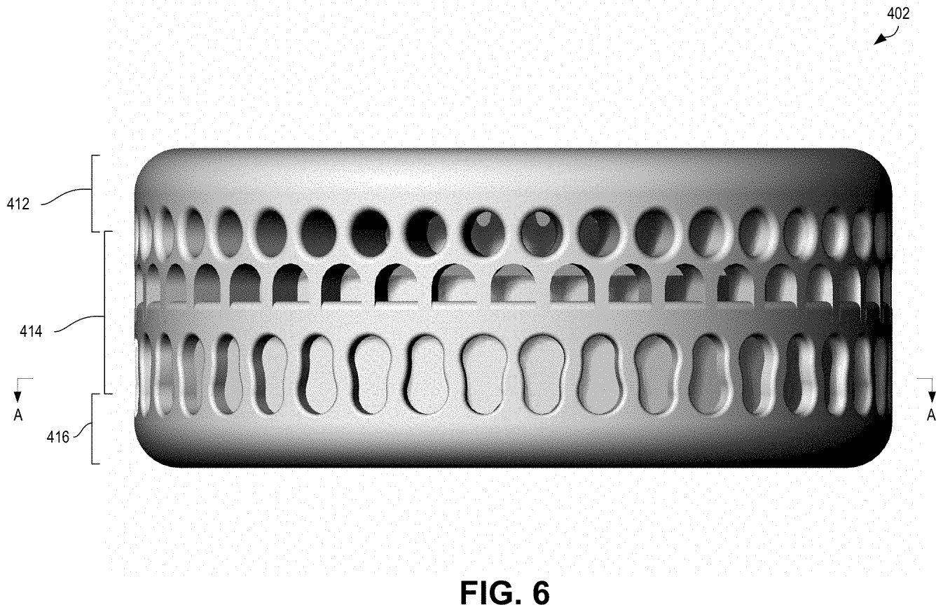

FIG. 6 is a side, elevation view of the round collection device 402 of FIG. 4 according to certain aspects of the present disclosure. The collection device 402 can include a collection zone 412, a filtration zone 414, and a processing zone 416.

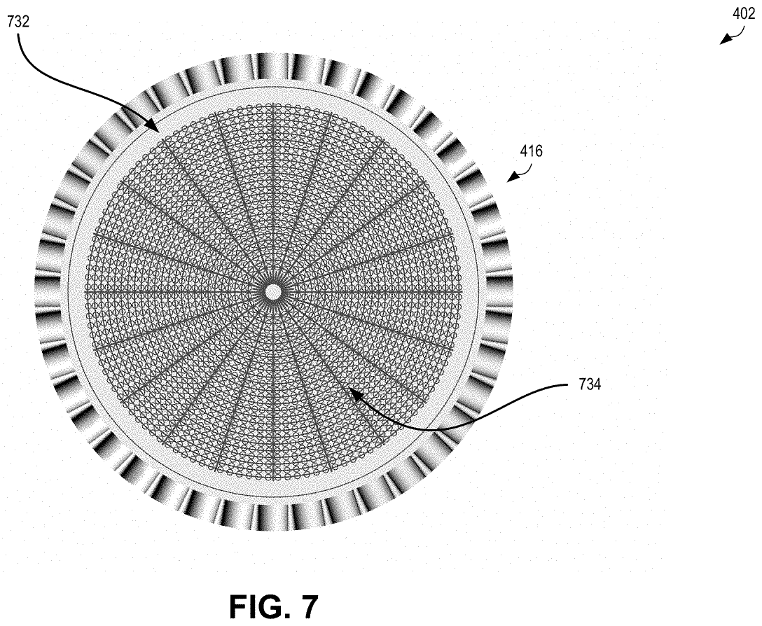

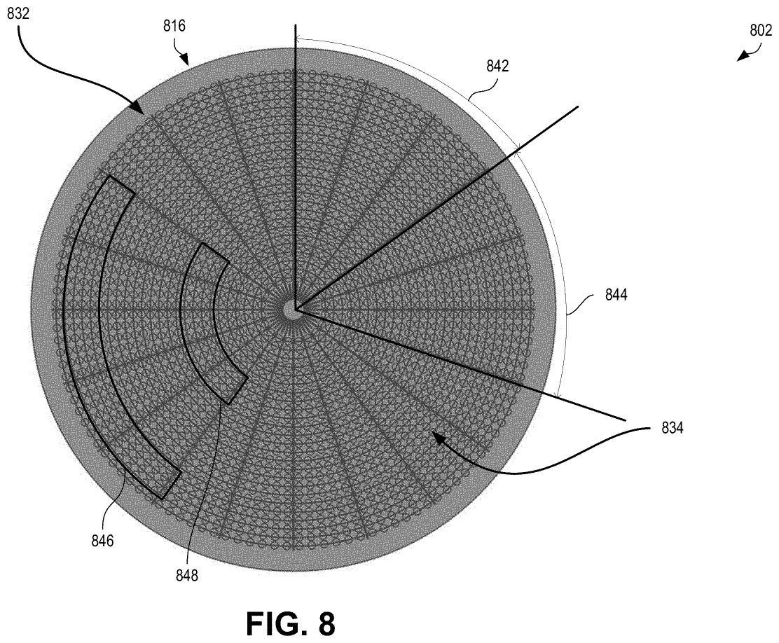

FIG. 7 is a top cutaway view of the round collection device 402 of FIG. 4 taken along line A:A of FIG. 6 according to certain aspects of the present disclosure. The processing zone 416 of the collection device 402 is depicted. Numerous wells 734 of the array of wells 732 are shown. The wells 734 is depicted arranged radially about the center of the collection device 402, however they can also be otherwise arranged. Each of the wells 734 can be approximately the same size (e.g., same diameter). In some cases, however, some wells can be larger than others.

FIG. 8 is a top view of an array of wells 832 of a processing zone 816 of a collection device 802 according to certain aspects of the present disclosure. The array of wells 832 can include a number of individual wells 834. The wells 834 can be pre-loaded with materials (e.g., PCR reagents and/or DNA probes) according to assay desired to be performed. In some cases, the array of wells 832 can be separated into regions, wish each region containing a subset of wells of the array of wells 832 prepared for a respective particular assay.

In some cases, the regions can be contiguous regions, such as angular regions or radial regions. In other cases, the regions can be non-contiguous, comprising non-adjacent wells 834, such as randomly selected wells 834.

In some cases, an array of wells 832 can include a first angular region 842 and a second angular region 844, as well as any number of additional angular regions. Each angular region 842, 844 can include a number of wells 834 prepared with materials selected for that particular region 842, 844. All wells 834 within a particular angular region 842, 844 can perform identical assays.

In some cases, an array of wells 832 can include a first radial region 846 and a second radial region 848, as well as any number of additional radial regions. Each radial region 846, 848 can include a number of wells 834 prepared with materials selected for that particular region 846, 848. All wells 834 within a particular radial region 846, 848 can perform identical assays.

A single collection device 802 can include any number of and any combination of regions (e.g., combinations of radial, angular, and/or non-contiguous regions).

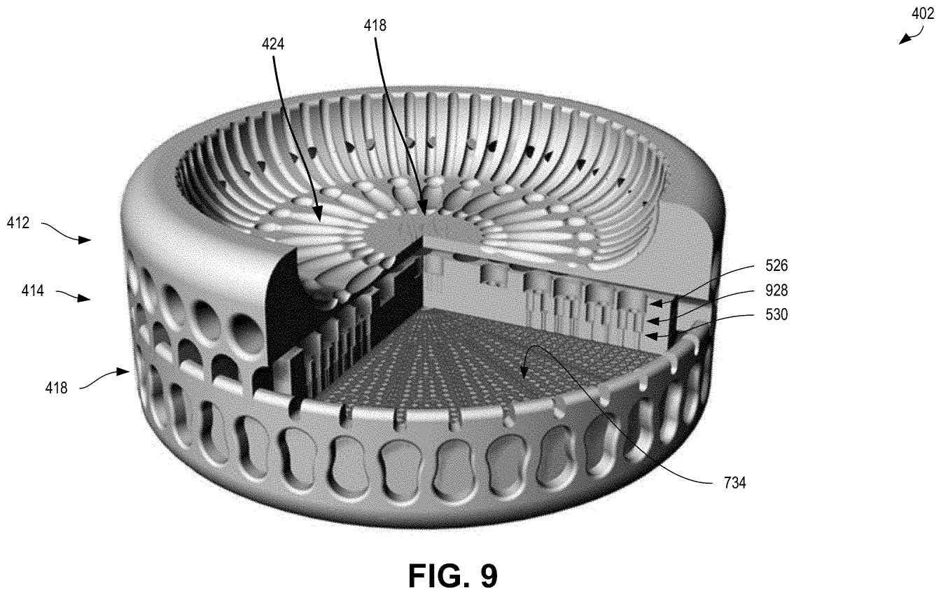

FIG. 9 is a partial cut-away axonometric view depicting the round collection device 902 of FIG. 4 according to certain aspects of the present disclosure. The collection device 402 can include a collection zone 412, a filtration zone 414, and a processing zone 416. The collection zone 412 can include a recess 438 suitable for placement of a digit, such as a finger or thumb. A needle array 418 can be seen in the center of the collection zone 412, although it may be placed off-center in some cases. Various apertures 424 of different shapes and dimensions are seen in the collection zone 412. The collection device 402 further includes optional, external ornamental apertures and shapes.

Also visible are numerous proximal passageways 526, middle passageways 928, and distal passageways 530 of the filtration zone 414. As seen in FIG. 8, the passageways 526, 928, 530 fluidly couple the collection zone 412 to the wells 734 of the processing zone 418.

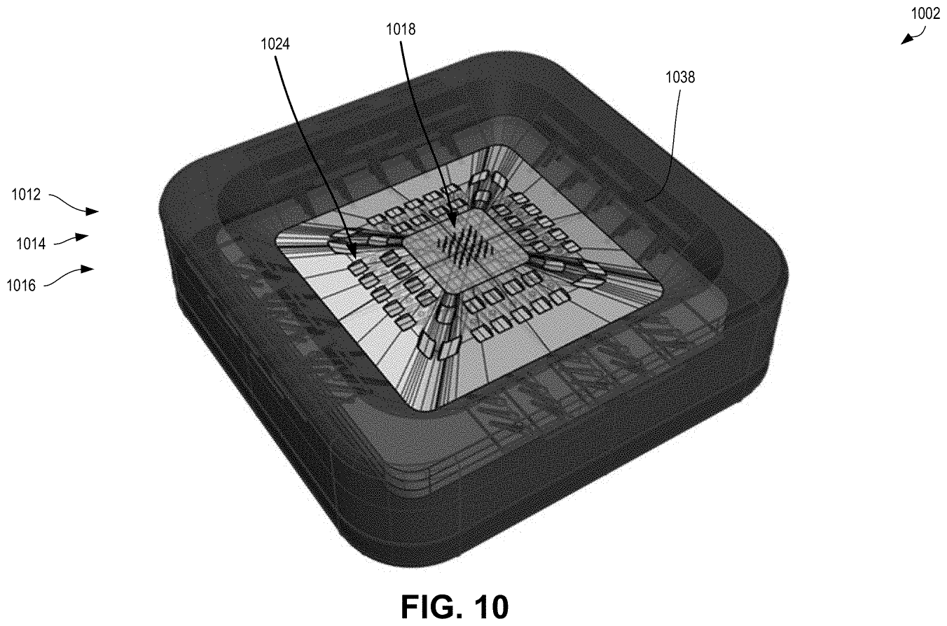

FIG. 10 is a partially see-through axonometric view depicting a square collection device 1002 according to certain aspects of the present disclosure. The collection device 1002 can include a collection zone 1012, a filtration zone 1014, and a processing zone 1016. The collection zone 1012 can include a recess 1038 suitable for placement of a digit, such as a finger or thumb. A needle array 1018 can be seen in the center of the collection zone 1012, although it may be placed off-center in some cases. Various apertures 1024 of different shapes and dimensions are seen in the collection zone 1012. The collection device 1002 further includes optional, external ornamental apertures and shapes.

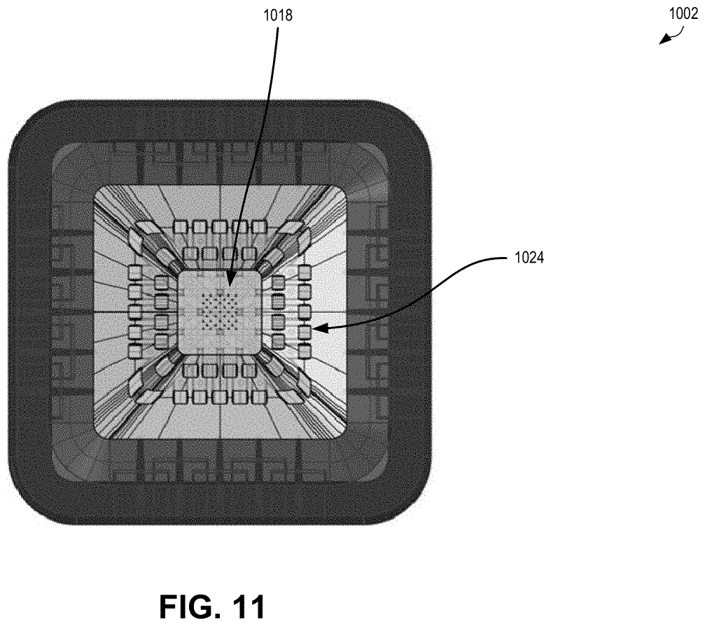

FIG. 11 is a partially see-through top view of the square collection device 1002 of FIG. 10 according to certain aspects of the disclosure. The needle array 1018 is seen centrally located. Numerous apertures 1024 in the collection zone are depicted.

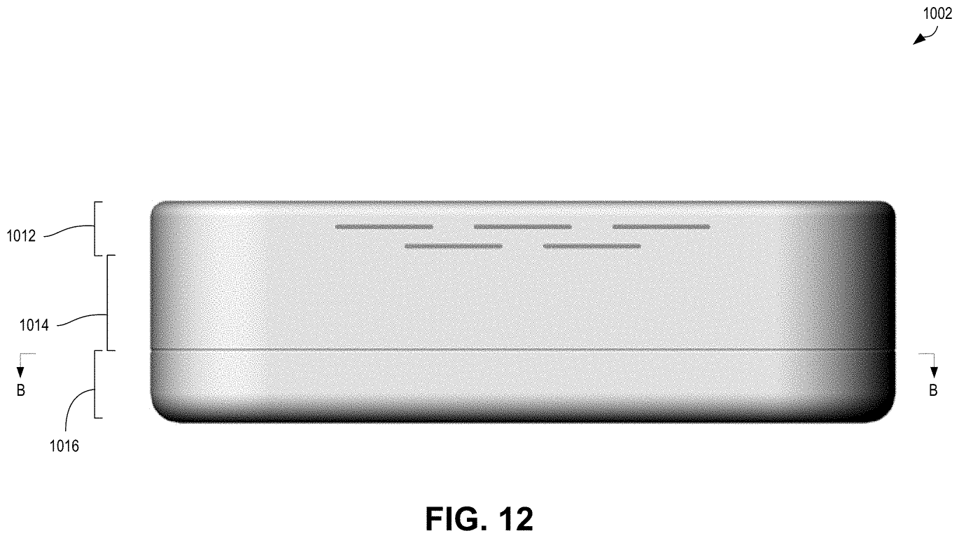

FIG. 12 is a side, elevation view of the square collection device 1002 of FIG. 10 according to certain aspects of the present disclosure. The collection device 1002 can include a collection zone 1012, a filtration zone 1014, and a processing zone 1016.

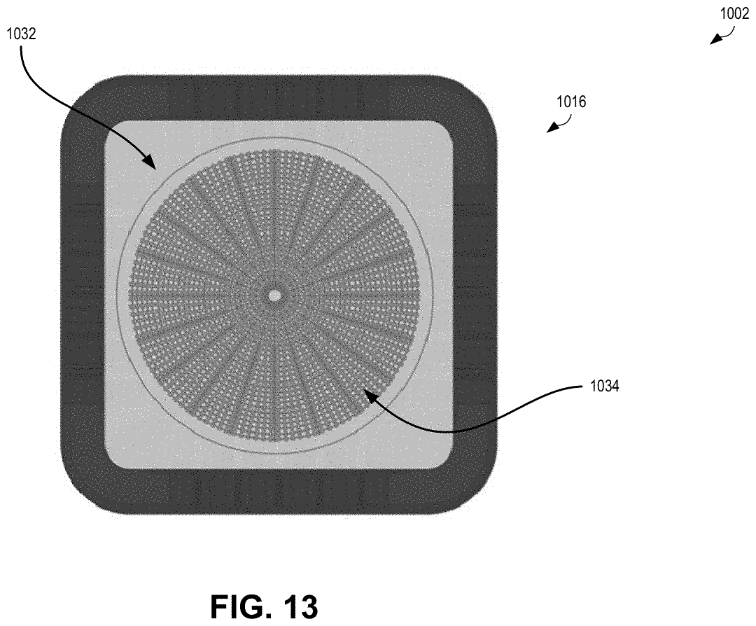

FIG. 13 is a top cutaway view of the square collection device 1002 of FIG. 10 taken along line B:B of FIG. 12 according to certain aspects of the present disclosure. The processing zone 1016 of the collection device 1002 is depicted. Numerous wells 1034 of the array of wells 1032 are shown. The wells 1034 is depicted arranged radially about the center of the collection device 1002, however they can also be otherwise arranged. Each of the wells 1034 can be approximately the same size (e.g., same diameter). In some cases, however, some wells can be larger than others.

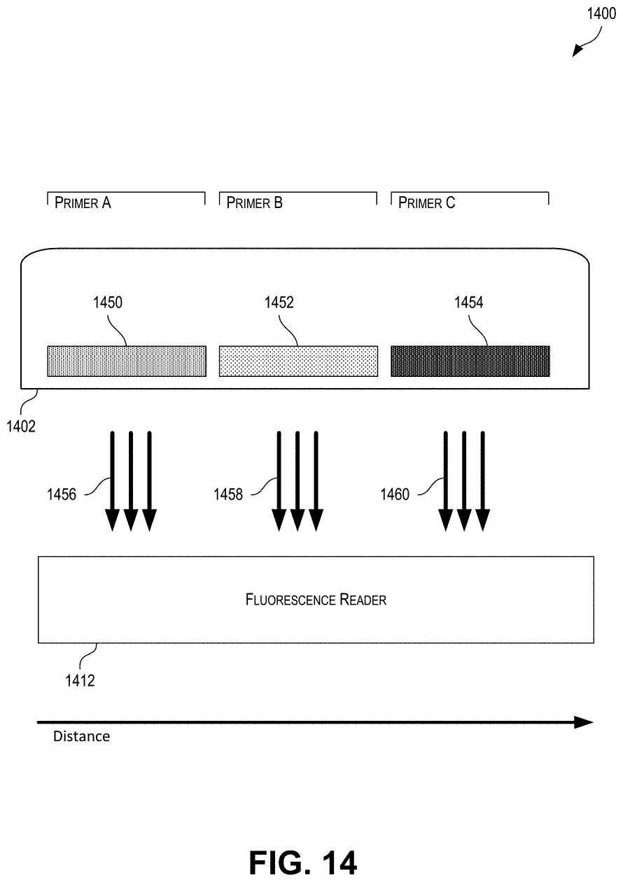

FIG. 14 is a schematic side view depicting an analysis system 1400 for detecting radiation emitted from a blood sample collection device 1402 prepared with multiple primers according to certain aspects of the present disclosure. The sample collection device 1402 can include any number of primers located within the wells of the array of wells of the processing zone of the collection device 1402. As depicted in FIG. 14, three primers, Primer A, Primer B, and Primer C, are located within first, second, and third regions 1450, 1452, 1454, respectively. Regions 1450, 1452, 1454 can be groupings of wells within the collection device 1402 that are each pre-loaded with primers A, B, and C, respectively. Regions 1450, 1452, 1454 can be located spatially offset from one another in any suitable fashion, such as spatially offset along a distance from an edge of the collection device (e.g., left edge as seen in FIG. 14).

During analysis, as the primers in each of the first, second, and third regions 1450, 1452, 1454 fluoresce, the fluorescence reader 1412 detects the light emitted from the primers. Because regions 1450, 1452, 1454 are spatially isolated from one another (e.g., longitudinally offset along the length of the collection device 1402), each can fluoresce first, second, and third light 1456, 1458, 1460, respectively that can be detected by spatially distinct portions of the fluorescent reader 1412 (e.g., portions of the fluorescent reader 1412 spatially offset along a distance from an edge of the fluorescent reader 1412, such as from the left edge as seen in FIG. 14). The fluorescence reader 1412 is thus able to distinctly identify the first light 1456 attributable to the region 1450 associated with Primer A, the second light 1458 attributable to the region 1452 associated with Primer B, and the third light 1460 attributable to the region 1454 associated with Primer C. The fluorescence reader 1412 can include suitable sensors for distinguishing the presence and/or amount of light across its longitudinal length and thus is able to individually distinguish first light 1456, second light 1458, and third light 1460.

In some cases, opaque (e.g., optically opaque) blockers (not shown) can be used to limit the amount of light 1456 emitted from region 1450 from spilling into the portion of the fluorescence reader 1412 aligned with region 1452.

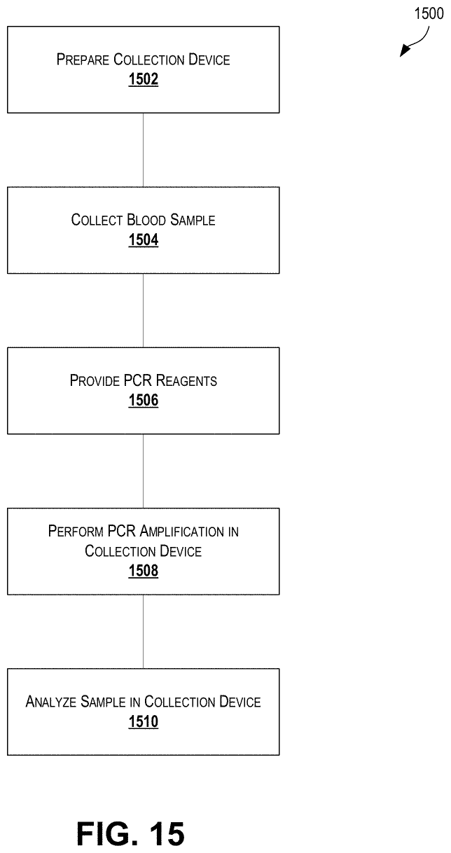

FIG. 15 is a flowchart depicting a process 1500 for collecting, amplifying, and analyzing a sample using a blood sample collection device according to certain aspects of the present disclosure. At block 1502, the collection device is prepared. Preparing the collection device can include removing sterility seals from collection zone or can include removing the collection device from a sterile container. In some cases, preparing the collection device can include putting materials, such as DNA primers, PCR reagents, lysing materials, or other materials, into the collection device.

At block 1504, the blood sample is collected. The blood sample can be collected by placing blood into the collection zone of the collection device or by having a patient place a digit onto a needle array of the collection device. After collecting the blood sample, the collection zone can be optionally sealed or secured to prevent sample contamination.

At optional block 1506, PCR reagents are placed into the wells if the wells do not yet have PCR reagents. In such cases, one or more of the apertures of the collection zone or alternate inlets may be left unsealed after block 1504 for insertion of the PCR reagents. In some case, other materials, such as lysing agents, can be added during block 1506. After adding the PCR reagents, the collection zone and/or alternate inlets can be optionally sealed.

At block 1508, PCR amplification is performed within the collection device. PCR amplification can include thermally cycling the collection device. In some cases, the entire collection device is placed in a PCR machine to undergo PCR amplification. In the PCR machine, the entire collection device can be subjected to thermal cycling to induce the various steps of PCR amplification. PCR amplification can thus be performed without the need to remove the sample from the collection device.

At block 1510, the samples in the collection device is analyzed. The samples can be analyzed by placing the collection device in, on, or adjacent an analysis machine, such as a machine including fluorescence sensors (e.g., optical sensors). Radiation (e.g., light) emitted by the samples can be emitted through an opening in the collection device, such as through a window.

In some cases, instead of a window, the processing zone can be separable from the remainder of the collection device and the processing zone can itself be placed in, on, or adjacent an analysis machine to allow the analysis machine to take readings from the wells from above the wells (e.g., where the filtration zone was prior to separation of the processing zone).

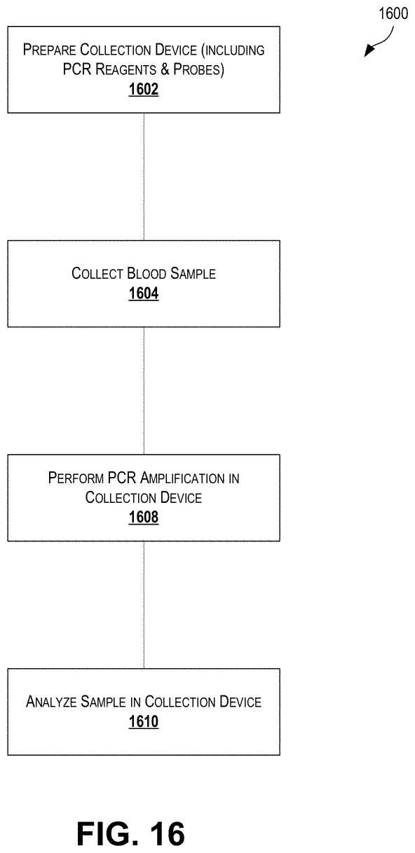

FIG. 16 is a flowchart depicting a process 1600 for collecting, amplifying, and analyzing a sample using a pre-loaded blood sample collection device according to certain aspects of the present disclosure. At block 1602, the collection device is prepared. Preparing the collection device can include removing sterility seals from collection zone or can include removing the collection device from a sterile container. Preparing the collection device can include pre-loading the collection device with PCR reagents and DNA primers (e.g., probes).

At block 1604, the blood sample is collected. The blood sample can be collected by placing blood into the collection zone of the collection device or by having a patient place a digit onto a needle array of the collection device. After collecting the blood sample, the collection zone can be optionally sealed or secured to prevent sample contamination.

At block 1608, PCR amplification is performed within the collection device. PCR amplification can include thermally cycling the collection device. In some cases, the entire collection device is placed in a PCR machine to undergo PCR amplification. In the PCR machine, the entire collection device can be subjected to thermal cycling to induce the various steps of PCR amplification. PCR amplification can thus be performed without the need to remove the sample from the collection device.

At block 1610, the samples in the collection device is analyzed. The samples can be analyzed by placing the collection device in, on, or adjacent an analysis machine, such as a machine including fluorescence sensors (e.g., optical sensors). Radiation (e.g., light) emitted by the samples can be emitted through an opening in the collection device, such as through a window.

In some cases, instead of a window, the processing zone can be separable from the remainder of the collection device and the processing zone can itself be placed in, on, or adjacent an analysis machine to allow the analysis machine to take readings from the wells from above the wells (e.g., where the filtration zone was prior to separation of the processing zone).

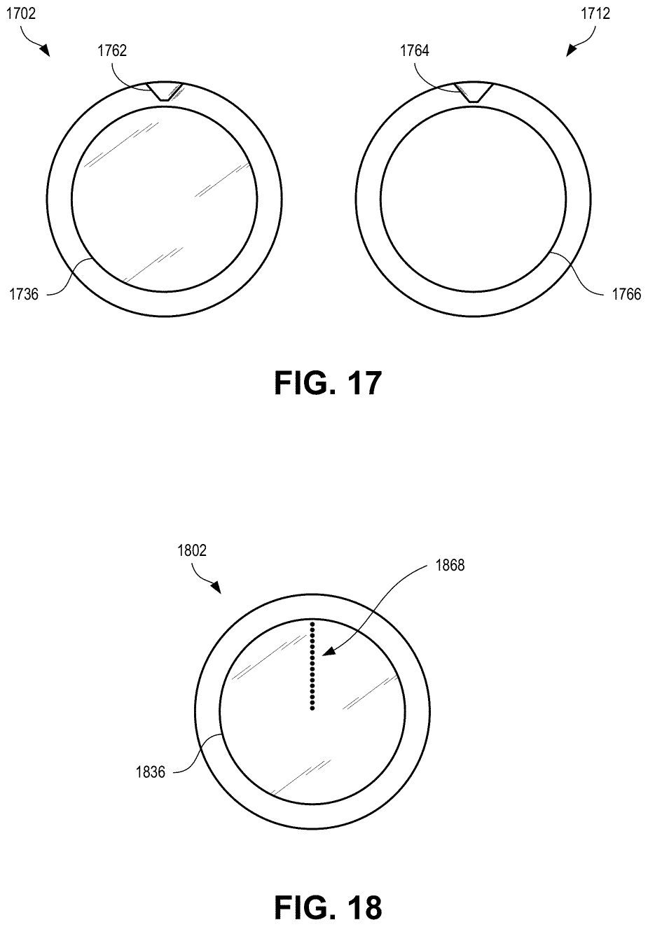

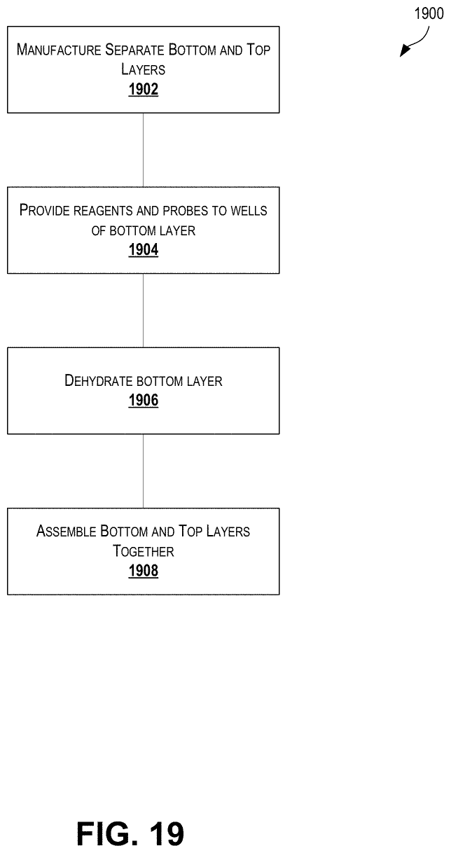

FIG. 17 is a combination image depicting a bottom view of a collection device 1702 and a top view of an analysis device 1712 according to certain aspects of the present disclosure. The collection device 1702 can include a window 1736 through which wells can be visible. To ensure desired alignment/orientation, such as when using an array of wells including various regions having different DNA probes, the collection device can include a key 1762 designed to fit a keyway 1764 of the analysis device 1712. The analysis device 1712 can include an observation window 1766 through which the analysis device 1712 takes readings of the wells of the collection device 1702.

In some cases, instead of a key 1762 and keyway 1764, a visual indicator or another mechanical feature can be used to ensure correct alignment/orientation. For example, opposing magnets in the collection device 1702 and analysis device 1712 can be used to ensure correct alignment/orientation.

FIG. 18 is a bottom view of a collection device 1802 featuring a recognizable pattern 1868 according to certain aspects of the present disclosure. Collection device 1802 can be placed on an analysis device with any alignment/orientation. Instead of relying on proper alignment and/or orientation, collection device 1802 can include a recognizable pattern 1868 visible to the analysis device. Upon reading the recognizable pattern 1868, software in the analysis device can automatically correct for any misalignment or incorrect orientation. In some cases, the analysis device may be agnostic to the recognizable pattern 1868, and instead a piece of analysis software can later be used to automatically correct for any misalignment or incorrect orientation. The recognizable pattern 1868 can be printed on the collection device 1802 or can be otherwise incorporated into the collection device 1802. In some cases, the recognizable pattern 1868 can include a number of wells prepared to put off a particular emission. Such wells can be sealed so that no blood cells flow therein.

FIG. 19 is a flowchart depicting a process 1900 of manufacturing a collection device according to certain aspects of the present disclosure. At block 1902, separate bottom and top layers of the collection device can be manufactured. The bottom layer can include the wells of the collection device (e.g., the processing zone). The top layer can include the collection zone and/or filtration zone of the collection device.

At block 1904, reagents and/or probes can be provided to the wells of the bottom layer. Reagents can include PCR reagents and any other reagents necessary for the particular assay. In some cases, probes can be provided to the wells. In some cases, probes can be dispersed amongst the wells generally. In some cases, distinct probes can be selectively provided to spatially isolated groups of wells to facilitate multiplexed assays. The reagents and/or probes can be immobilized within the well.

At block 1906, the bottom layer can be dehydrated. Dehydration can facilitate storage until the collection device is used.

At block 1908, the bottom layer and top layer are assembled together. The bottom layer and top layer can be secured together using any suitable technique, such as mechanical coupling, adhesives, welding, or other such techniques.

The foregoing description of the embodiments, including illustrated embodiments, has been presented only for the purpose of illustration and description and is not intended to be exhaustive or limiting to the precise forms disclosed. Numerous modifications, adaptations, and uses thereof will be apparent to those skilled in the art.

As used below, any reference to a series of examples is to be understood as a reference to each of those examples disjunctively (e.g., “Examples 1-4” is to be understood as “Examples 1, 2, 3, or 4”).

Example 1 is a sample collection device comprising a collection zone for accepting a blood sample; an array of wells for entrapping blood cells from the blood sample; and a filtration zone including a plurality of passageways coupling the collection zone to the array of wells, wherein an average diameter of the plurality of passageways decreases from a cross section adjacent the collection zone to a cross section adjacent the array of wells.

Example 2 is the sample collection device of example 1, wherein the collection zone includes a needle array to facilitate drawing the blood sample.

Example 3 is the sample collection device of examples 1 or 2, further comprising a window located at or adjacent the array of wells for visually exposing the blood cells entrapped within the array of wells to an exterior of the device.

Example 4 is the device of examples 1-3, wherein interior surfaces of the array of wells are made of plasmonic materials coated with a protective layer.

Example 5 is the device of example 4, wherein the protective layer is made of SiO2 or Al2O3.

Example 6 is the device of examples 1-5, further comprising polymerase chain reaction reagents preloaded into each well of the array of wells.

Example 7 is the device of examples 1-6, further comprising a plurality of DNA primers, wherein the plurality of DNA primers comprises a first primer preloaded into a first set of wells of the array of wells and a second primer preloaded into a second set of wells of the array of wells.

Example 8 is a method, comprising introducing a blood sample to a collection zone of a collection device, wherein introducing the blood sample to the collection zone results in blood cells of the blood sample being directed to an array of wells of the collection device through passageways of a filtration zone of the sample collection device; amplifying genetic material of the blood cells within the array of wells; and measuring emitted radiation from the collection device.

Example 9 is the method of example 8, wherein introducing the blood sample includes using a needle array of the collection zone of the collection device to facilitate extraction of the blood sample.

Example 10 is the method of examples 8 or 9, wherein the internal surface of each well of the array of wells includes plasmonic materials coated with a protective layer.

Example 11 is the method of example 10, wherein the protective layer is made of SiO2 or Al2O3.

Example 12 is the method of examples 8-11, wherein the array of wells include polymerase chain reaction reagents pre-deposited therein, and wherein amplifying genetic material includes thermally cycling the collection device to induce polymerase chain reactions within the array of wells.

Example 13 is the method of examples 8-12, wherein introducing the blood sample includes passing blood cells through sequentially narrower passageways of the filtration zone of the sample collection device and into the array of wells.

Example 14 is the method of examples 8-13, further comprising preloading the array of wells with a plurality of deoxyribonucleic acid (DNA) primers, wherein preloading the array of wells with the DNA primers includes preloading a first primer into a first subset of wells of the array of wells and preloading a second primer into a second subset of wells of the array of wells.

Example 15 is a system, comprising a collection device comprising: a collection zone for accepting a blood sample; an array of wells for entrapping blood cells from the blood sample; and a filtration zone including a plurality of passageways coupling the collection zone to the array of wells, wherein an average diameter of the plurality of passageways decreases from a cross section adjacent the collection zone to a cross section adjacent the array of wells. The system also includes a polymerase chain reaction (PCR) machine for accepting the collection device and performing PCR amplification on the collection device; and an analysis machine for accepting the collection device and measuring an optical signal emitted from the collection device. The PCR machine and the analysis machine can be collocated or located in separate enclosures.

Example 16 is the system of example 15, wherein the collection zone includes a needle array for facilitating drawing the blood sample.

Example 17 is the system of examples 15 or 16, wherein the interior surface of each well of the array of wells is made of plasmonic materials coated with a protective layer.

Example 18 is the system of example 17, wherein the protective layer is made of SiO2 or Al2O3.

Example 19 is the system of examples 15-18, wherein the collection device further comprises PCR reagents preloaded into each well of the array of wells.

Example 20 is the system of examples 15-19, wherein the collection device further comprises a plurality of DNA primers, wherein the plurality of DNA primers comprises a first primer preloaded into a first subset of wells of the array of wells and a second primer preloaded into a second subset of wells of the array of wells.

What is claimed is:

1. A sample collection device comprising:
a collection zone for accepting a blood sample;
an array of wells for entrapping blood cells from the blood sample; and
a filtration zone including a plurality of passageways coupling the collection zone to the array of wells, wherein an average diameter of the plurality of passageways decreases from a cross section adjacent the collection zone to a cross section adjacent the array of wells.

2. The sample collection device of claim 1, wherein the collection zone includes a needle array to facilitate drawing the blood sample.

3. The sample collection device of claim 1, further comprising a window located at or adjacent the array of wells for visually exposing the blood cells entrapped within the array of wells to an exterior of the device.

4. The device of claim 1, wherein interior surfaces of the array of wells are made of plasmonic materials coated with a protective layer.

5. The device of claim 4, wherein the protective layer is made of SiO2 or A1203.

6. The device of claim 1, further comprising polymerase chain reaction reagents preloaded into each well of the array of wells.

7. The device of claim 1, further comprising a plurality of DNA primers, wherein the plurality of DNA primers comprises a first primer preloaded into a first set of wells of the array of wells and a second primer preloaded into a second set of wells of the array of wells.

8. A method, comprising:
introducing a blood sample to a collection zone of a collection device, wherein introducing the blood sample to the collection zone results in blood cells of the blood sample being directed to an array of wells of the collection device through passageways of a filtration zone of the sample collection device;
amplifying genetic material of the blood cells within the array of wells; and
measuring emitted radiation from the collection device.

9. The method of claim 8, wherein introducing the blood sample includes using a needle array of the collection zone of the collection device to facilitate extraction of the blood sample.

10. The method of claim 8, wherein the internal surface of each well of the array of wells includes plasmonic materials coated with a protective layer.

11. The method of claim 10, wherein the protective layer is made of SiO₂ or Al₂O₃.

12. The method of claim 8, wherein the array of wells include polymerase chain reaction reagents pre-deposited therein, and wherein amplifying genetic material includes thermally cycling the collection device to induce polymerase chain reactions within the array of wells.

13. The method of claim 8, wherein introducing the blood sample includes passing blood cells through sequentially narrower passageways of the filtration zone of the sample collection device and into the array of wells.

14. The method of claim 8, further comprising preloading the array of wells with a plurality of deoxyribonucleic acid (DNA) primers, wherein preloading the array of wells with the DNA primers includes preloading a first primer into a first subset of wells of the array of wells and preloading a second primer into a second subset of wells of the array of wells.

15. A system, comprising:
a collection device comprising:
a collection zone for accepting a blood sample;
an array of wells for entrapping blood cells from the blood sample; and
a filtration zone including a plurality of passageways coupling the collection zone to the array of wells, wherein an average diameter of the plurality of passageways decreases from a cross section adjacent the collection zone to a cross section adjacent the array of wells;
a polymerase chain reaction (PCR) machine for accepting the collection device and performing PCR amplification on the collection device; and
an analysis machine for accepting the collection device and measuring an optical signal emitted from the collection device.

16. The system of claim 15, wherein the collection zone includes a needle array for facilitating drawing the blood sample.

17. The system of claim 15, wherein the interior surface of each well of the array of wells is made of plasmonic materials coated with a protective layer.

18. The system of claim 17, wherein the protective layer is made of SiO2 or A1203.

19. The system of claim 15, wherein the collection device further comprises PCR reagents preloaded into each well of the array of wells.

20. The system of claim 15, wherein the collection device further comprises a plurality of DNA primers, wherein the plurality of DNA primers comprises a first primer preloaded into a first subset of wells of the array of wells and a second primer preloaded into a second subset of wells of the array of wells.
